(19) United States
(12) Patent Application Publication
(10) Pub. No.: US 2004/0127817 A1
(43) Pub. Date: Jul. 1, 2004

(54) Blood collection set

(76) Inventors: Chang-Ming Yang, Miaoli (TW); Tzu-Lin Yang, Miaoli (TW)

Correspondence Address: TROXELL LAW OFFICE PLLC, SUITE 1404, 5205 LEESBURG PIKE, FALLS CHURCH, VA, 22041, US

(21) Appl. No.: 10/329,453

(22) Filed: Dec. 27, 2002

(51) Int. Cl. A61B 5/00

(52) U.S. Cl. 600/576

(57) ABSTRACT

A blood collection set comprises essentially a syringe, a syringe cover, a flexible tube and a test tube, wherein on the top of said syringe body, there is provided protrudingly with one or more fasteners; and wherein, on the syringe cover, there are provided with several gliding slots corresponding to the number and positions of said fasteners on said syringe, wherein, on both of the front and rear ends of said gliding slots, a protruding button is provided convex in a manner that a fastening portion is formed on the front end and the rear end of said gliding slots, respectively, and wherein the base of the syringe cover is flat and is engaged with said lateral pieces of the syringe; wherein the front end of the flexible tube is connected with the rear end of the syringe, while a plastic needle is provided on the rear end of said flexible tube; and wherein an interposing structure is provided by sleeving on said flexible tube.

### BACKGROUND OF THE INVENTION

1. Field of the Invention

The invention relates to a blood collection set, and in particular, to a blood collection set characterized in that a syringe is engaged with a syringe cover and a plastic needle is used to replace the metal needle previously engaged at the rear end of a flexible tube to prevent medical practitioner from risk during operation.

2. Description of the Prior Art

Conventionally, a blood collection set is consisted of a syringe 1, a syringe cover 2, a flexible tube 3, and a test tube 4, as depicted in FIG. 26, wherein, said syringe 1 is provided with several lateral pieces 12 extended from each sidewall of a syringe body 11, respectively, and in front of said syringe body 11, an injection needle 111 is provided thereto. The syringe cover 2 is in a cylindrical form having an accommodating aperture at its base. The flexible tube 3 is connected at its front end with the rear end of the syringe 1, while the rear end of the flexible tube 3 is connected with an injection needle 111. Around the injection needle 111, there is sleeved on with a sheath 6 made of an elastic material. Alternatively, the injection needle 111 that is connected with the flexible tube 3 may be sleeved on with an outer thimble 5. As the conventional blood collection set is used, the syringe cover 2 should be removed at first from the syringe 1 and an injection is performed by inserting the injection needle 111 into the body of a subject, and then the injection needle 111 that is connected to the rear end of the flexible tube 3 is punctured into the test tube 4. At this moment, the sheath 6 sleeved on the outer periphery of injection needle 111 is forced to contract back such that the injection needle 111 breaks through the sheath 6 and punctures into the test tube 4. When the test tube 4 is to be replaced, it needs just to pull the injection needle 111 out of the test tube 4, while the sheath 6 will recover to its original shape under its own elastic property and encloses the injection needle 111, and, thereafter, the injection needle 111 is needed just to puncture into another test tube 4. When the blood collection is accomplished, the injection needle 111 connected to the rear end of the flexible tube 3 can be pulled out of the test tube 4, and the syringe 1 is drawn out of the subject, covered with the syringe cover 2, and then the syringe 1, the syringe cover 2 and the flexible tube 3 are discarded all together. However, in the course of the blood collection by using the above-described conventional blood collection set, since the syringe cover 2 should be removed from the syringe 1 at first before injecting, after completion of the blood collection, the medical practitioner should return the syringe cover 2, and just at this time, he (she) will be subject to be hurt by the injection needle if carelessly handled, and hence is susceptible to be infected. Furthermore, although there is a sheath 6 sleeved on the periphery of the injection needle 111 that is connected to the rear end of the flexible tube 3, unfortunately, since the sheath 6 is made of an elastic material, in the course of the blood collection or after discarding the flexible tube 3, the injection needle 111 might puncture through the sheath and hence hurts and causes infection to the medical practitioner or the cleanser.

Accordingly, the conventional art mentioned above has still many disadvantages and is not designed perfectly such that it needs to be improved immediately.

In view of the various disadvantages associated with the prior art blood collection set, the inventor of this application had devoted to improve it and, after studying intensively for many years, accomplished successfully the present blood collection set.

### SUMMARY OF THE INVENTION

Accordingly, one object of the invention is to provide a blood collection set characterized in that the syringe is engaged with a syringe cover.

Another object of the invention is to provide a blood collection set characterized in that it can prevent from risk of being hurt for the medical practitioner in the course of blood collection or for the cleanser in cleansing.

The blood collection set that can accomplishes the above-described objects according to the invention is consisted of:

a syringe, provided with several lateral pieces each extending from the base of the syringe body, respectively, wherein said syringe body is fitted with an injection needle at the front end thereof, and is provided protrudingly with one or more fasteners at the top thereof;

a syringe cover, provided with several gliding slots thereon corresponding to the number and positions of said fasteners on said syringe, said gliding slots being fastened on said fasteners on said syringe, wherein on both of the front and rear ends of said gliding slots, a protruding button is provided in a manner that a fastening portion is formed on the front end and the rear end of said gliding slots, respectively, and wherein the base of the syringe cover is flat and is engaged with said lateral pieces of the syringe;

a flexible tube, the front end of said flexible tube being connected with the rear end of said syringe, while the rear end of said flexible tube being connected with a plastic needle, wherein on said flexible tube, a interposing structure is sleeved on; and

a test tube, provided with a test tube cap that is made of an elastic material and is sleeved on the opening of said test tube.

### BRIEF DESCRIPTION OF THE DRAWINGS

The drawings disclose an illustrative embodiment of the present invention which serves to exemplify the various advantages and objects hereof, and are as follows:

FIG. 1 is a three-dimensional exploded view of the blood collection set according to the invention;

FIG. 2 is the three-dimensional out-looking view of the blood collection set depicted in FIG. 1;

FIG. 3 is the three-dimensional out-looking view of an embodiment of the blood collection set depicted in FIG. 1;

FIG. 4 is a partial enlarged view of the syringe and the syringe cover;

FIG. 5 is a three-dimensional exploded view of the syringe and the syringe cover;

FIG. 6 is a schematic view showing the action of the syringe and the syringe cover;

FIG. 7 is the three-dimensional exploded view of the interposing element;

FIG. 8 is a schematic view showing the action of the first embodiment of said interposing element;

FIG. 9 is a schematic view showing the action of the second embodiment of said interposing element;

FIG. 10 is a three-dimensional exploded view of the first embodiment of the syringe and the syringe cover;

FIG. 11 is a three-dimensional out-looking view of the second embodiment of the syringe and the syringe cover;

FIG. 12 is a three-dimensional out-looking view of the third embodiment of the syringe and the syringe cover;

FIG. 13 is a three-dimensional exploded view of the third embodiment of the syringe and the syringe cover;

FIG. 14 is a three-dimensional out-looking view of the fourth embodiment of the syringe and the syringe cover;

FIG. 15 is a three-dimensional exploded view of the fourth embodiment of the syringe and the syringe cover;

FIG. 16 is a three-dimensional exploded view of the first embodiment of the test tube and the guiding tube;

FIG. 17 is the cross-section side view of the first embodiment of the test tube;

FIG. 18 is the cross-section side view of the second embodiment of the test tube;

FIG. 19 is the three-dimensional exploded view of the third embodiment of the test tube;

FIG. 20 is the three-dimensional exploded view of the fourth embodiment of the test tube;

FIG. 21 is a cross-section side view of the fifth embodiment of the test tube;

FIG. 22 is a schematic side view showing the action of the fifth embodiment of the test tube;

FIG. 23 is a three-dimensional exploded view of the sixth embodiment of the test tube;

FIG. 24 is a cross-section side view of the sixth embodiment of the test tube;

FIG. 25 is a three-dimensional exploded view of the fifth embodiment of the syringe and the syringe cover; and

FIG. 26 is a three-dimensional exploded view of a conventional blood collection set.

### DETAILED DESCRIPTION OF THE PREFERRED EMBODIMENT

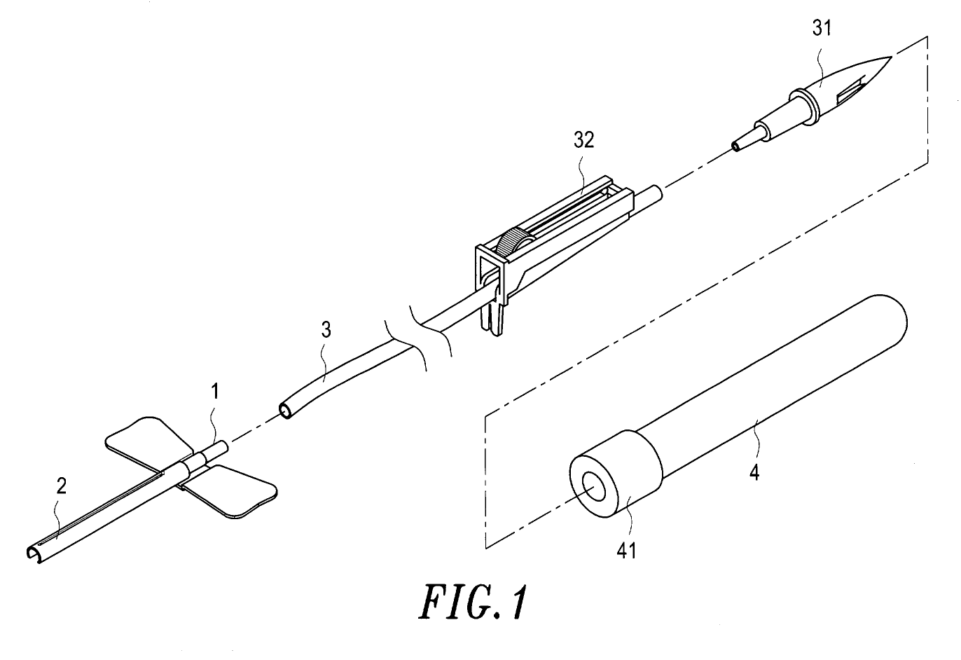

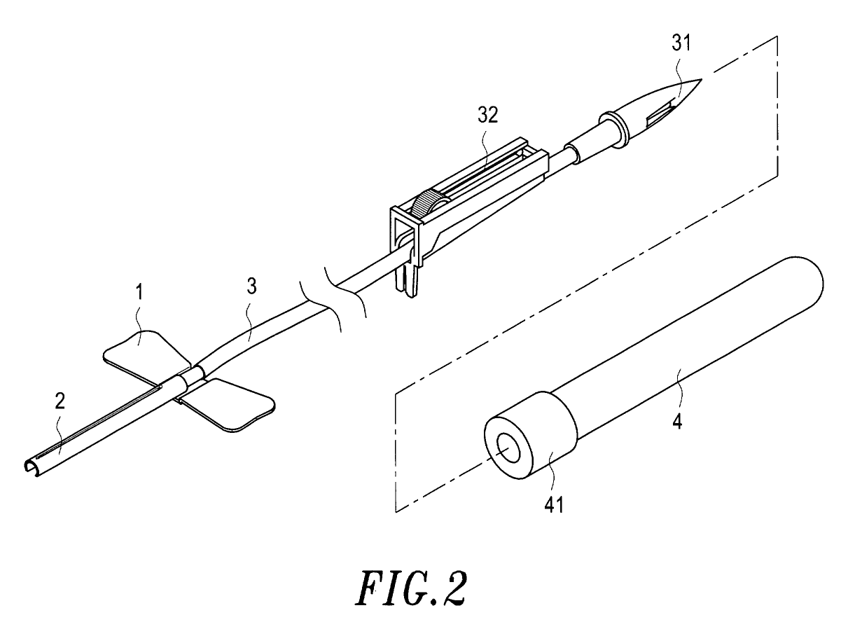

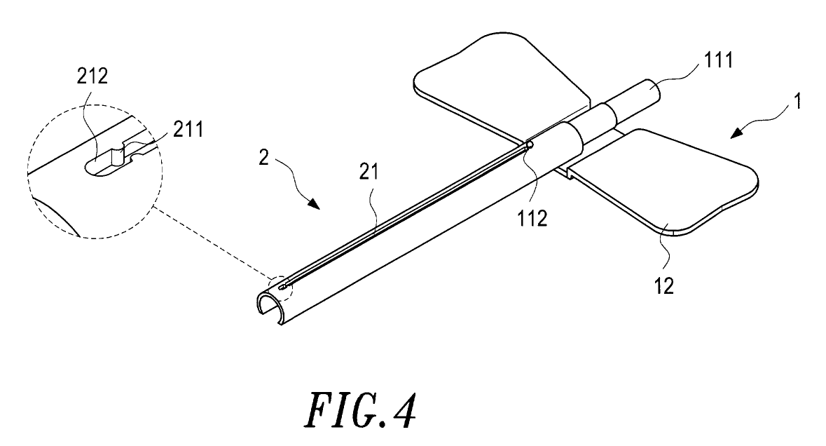

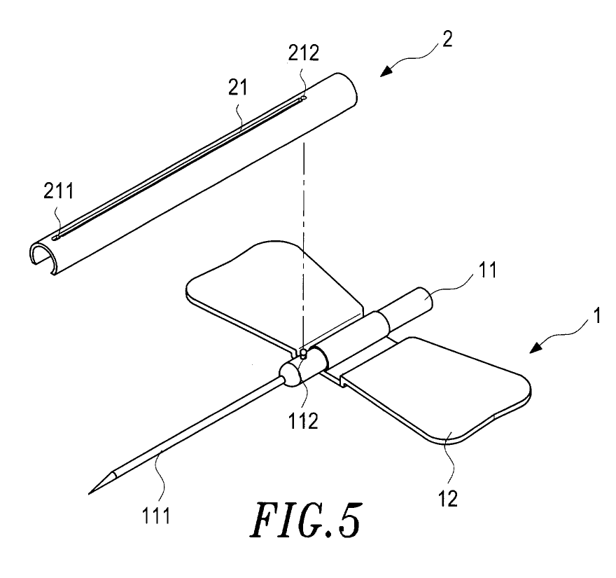

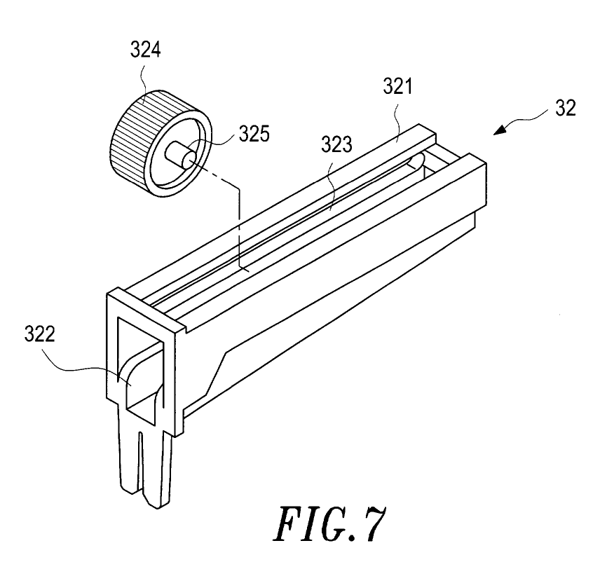

Referring to FIGS. 1, 2, 4, 5, and 7, the blood collection set provided by the invention is consisted essentially of:

a syringe 1, provided with several lateral pieces 12 each extending from the base of the syringe body 11, respectively, wherein said syringe body 111 is fitted with an injection needle 111 at the front end thereof, and is provided protrudingly with one or more fasteners 112 at the top thereof;

a syringe cover 2, provided with several gliding slots 21 thereon corresponding to the number and positions of said fasteners 112 on said syringe 1, wherein on both of the front and rear ends of said gliding slots 21, a protruding button 211 is provided in a manner that a fastening portion 212 is provided on the front end and the rear end, respectively, and wherein the base of the syringe cover 2 is flat and is engaged with said lateral pieces 12 of the syringe 1;

a flexible tube 3, the front end of said flexible tube 3 being connected with the rear end of said syringe 1, while the rear end of said flexible tube being connected with a plastic needle, wherein on said flexible tube 3, a interposing structure 32 is sleeved on, said interposing structure being consisted of a main body 321 and a holding element 324, wherein, on both sides of said holding element 324, a shaft lever 325 is extended there from; wherein, within said main body 321, there is provided concave an accommodating slot 322 that has a slope for said flexible tube to extend through, and on each of both wall surfaces of said accommodating slot 322, there is provided a moving slot 323 for positioning of said shaft lever 325 of said holding element 324; and

a test tube 4, provided with a test tube cap 41 that is made of an elastic material and is sleeved on the opening of said test tube 4;

wherein said syringe 1 is positioned within said gliding slot 21 of said syringe cover 2 via said fastener 112, and said fastener 112 comprises a spherical body provided on the top of a cylindrical post in a form that the outer diameter of said spherical body is larger than that of said cylindrical post; wherein said fastener 112 is gliding in said gliding slot 21 of said syringe cover 2 by means of said cylindrical post while said spherical body is fixed against the top surface of said gliding slot 21 in a manner that the syringe cover 2 can be prevent from detaching off said syringe 1; and alternatively, said fastener 112 can be provided at any desired position around the periphery of said syringe body 11 of said syringe 1′ while said plastic needle 31 provided on the rear end of said flexible tube 3 can puncture into said test tube cap 41.

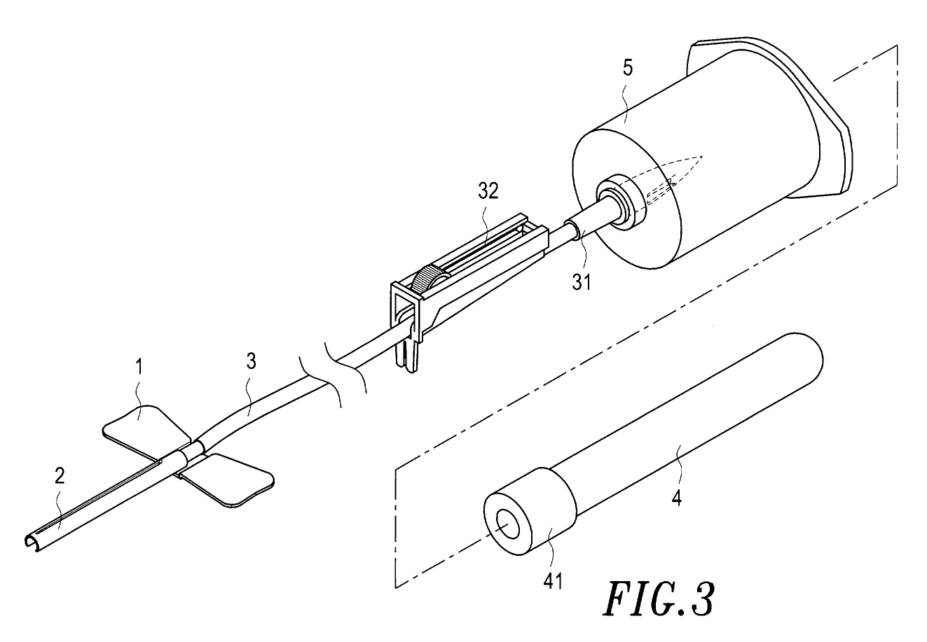

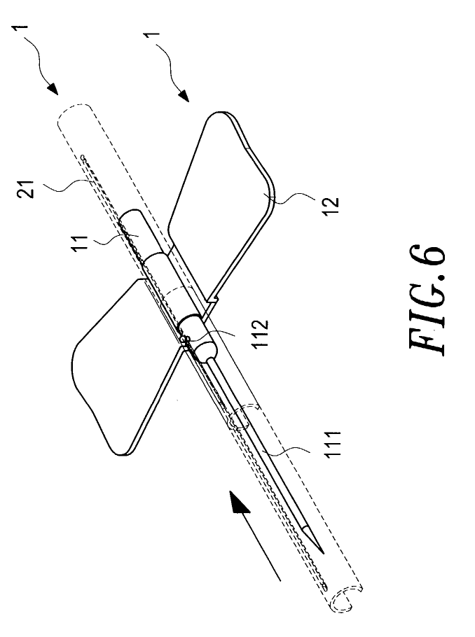

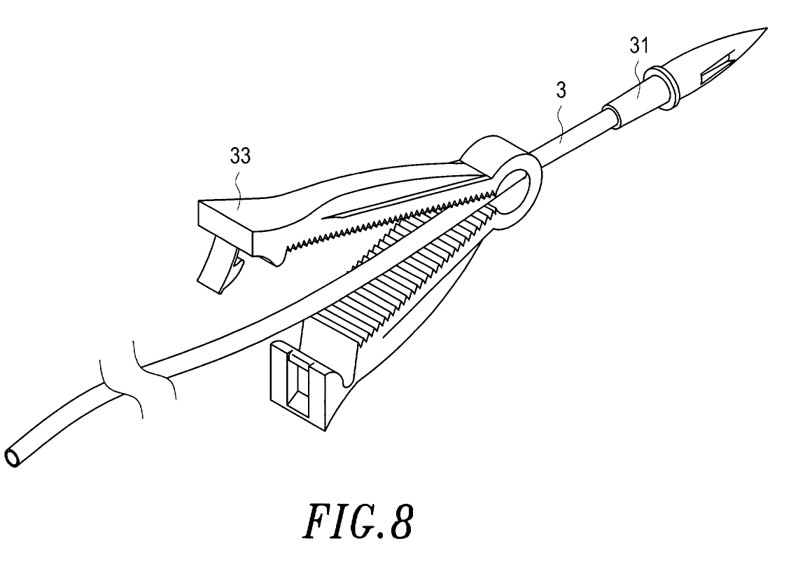

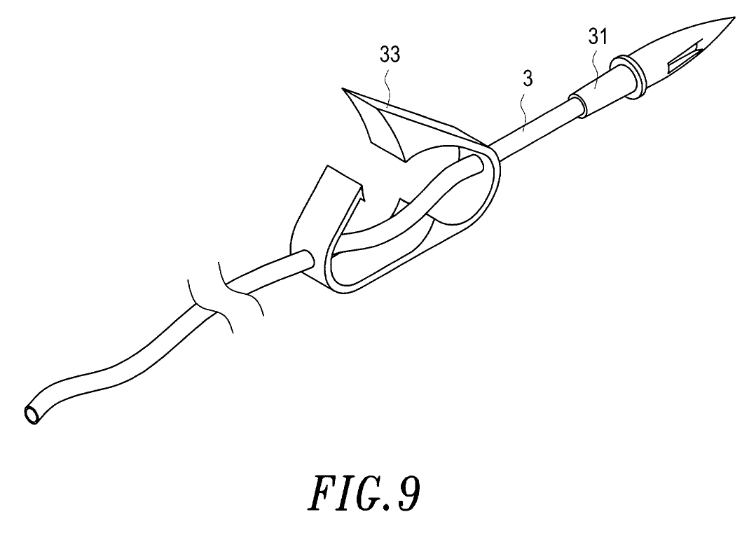

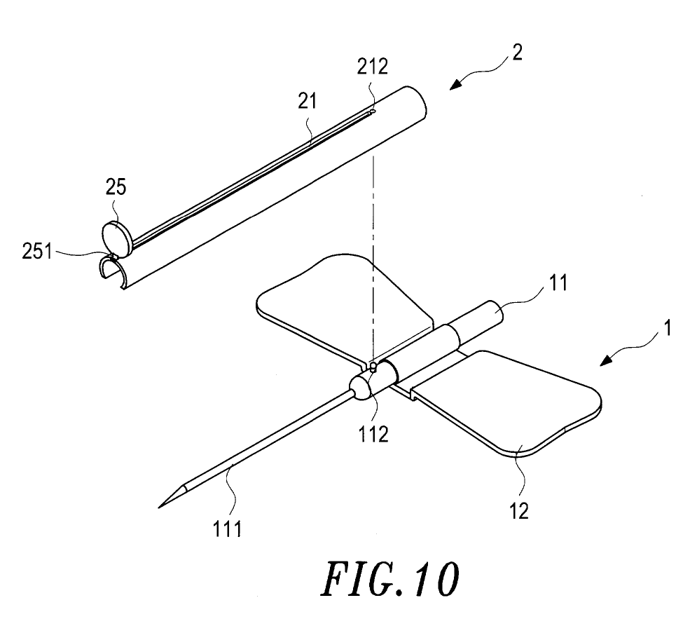

Referring to FIGS. 1, 2, 3, 6, 7, 8, 9, and 10, when the blood collection set according to the invention is not used, the syringe cover 2 covers originally over the outside of the injection needle 111, the gliding slot 21 on the top of the syringe cover 2 is fastened on the fastener 112 of the syringe 1 and the fastener 112 of the syringe 1 is accommodated in a fastening portion 212 formed by a protruding button 211 at the rear end of the gliding slot 21. When the blood collection set according to the invention is to be used, it needs just push the syringe cover 2 backwardly such that the fastener 112 of the syringe 1 detaches from the fastening portion 212 at the rear end of the gliding slot 21, and the syringe cover 2 can slide over the syringe 1 by means of the gliding slot 21 and the fastener 112. When the syringe cover 2 slides to the rear end of the syringe 1, the protruding button 211 on both ends of the gliding slot 21 will stop the fastener 112 and returns it back into the fastening portion 212 at the front of the gliding track. Under this circumstance, the injection needle 111 protruding out of the syringe 1 can be used to puncture into a subject. At this moment, the plastic needle 31 that is provided at the rear end of the flexible tube 3 connected to the rear end of the syringe 1 will insert into the test tube cap 41 of the test tube 4 in a manner that the blood collected will flow from the syringe 1 into the test tube 4. Thereafter, in case for replacing test tube, it can be done simply by pushing the holding element 324 of the interposing structure 32 to move the shaft lever 325 in the moving slot 323 of the main body 321 in a manner that the holding element 324 can compress the flexible tube 3 that has passed through the accommodating slot 322 of the main body 321 and that the blood can not pass through, and then the plastic needle 31 is drawn out of the test tube 4, inserted in a new test tube 4, and, the holding element 324 of the interposing structure 32 is pushed to its original position such that the holding element 324 can no longer compress the flexible tube 3 and hence allow the blood flow through. After completion of blood collection, it needs just push the syringe cover 2 back to its original position, and fastens it through fixing the fastener 112 with the fastening portion 212 at the rear end of the gliding slot 21. As described above, in use of the blood collection set according to the invention, by virtue of the engagement of the syringe 1 with the syringe cover 2, in the course of the injection, the syringe cover 2 will not detach from the syringe 1, and, in addition, by replacing a plastic needle 31 for a conventional injection needle 111, the medical practitioner shall never be punctured accidentally by the injection needle in the course of returning the syringe cover 2 and hence will be not infected. Furthermore, an outer thimble 5 can be provided over the plastic needle 31. Moreover, the interposing structure 32 can be substituted with a clipping element 33. Further, at the front end of the syringe cover 2, a cap 25 can be provided to connect through a connecting element 251. Before and after using of the blood collection set according to the invention, the syringe 1 can be covered with the cap 25 to prevent the injection needle 111 at the front end of the syringe 1 from piercing through to hurt the medical practitioner or the cleanser.

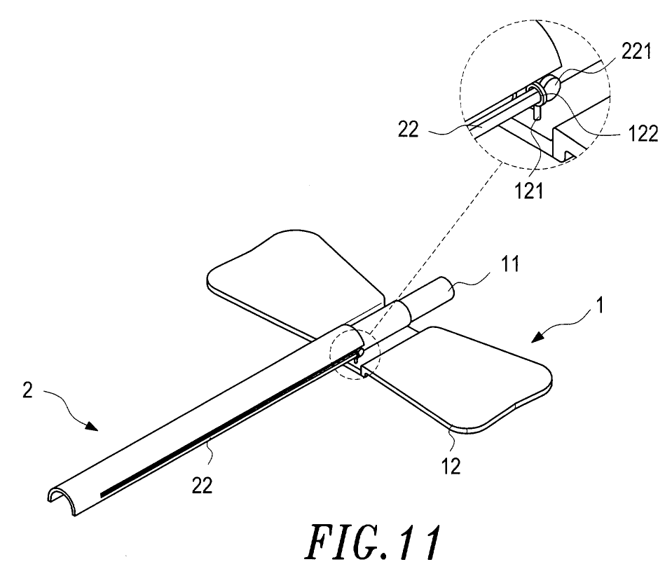

Referring to FIG. 11, in one embodiment of the blood collection set according to the invention, guiding structures 121 having an aperture 122 can be provided at the front end of both lateral sides of the syringe body 11. Further, on both sides of the syringe cover 2, there are provided guiding tracks 22 that extend backwardly therefrom and through the aperture 122 of the guiding structure 121. On the rear end of each guiding track 22, there is provided with a protruding sphere 221 whose outer diameter is larger than the diameter of the aperture 122 on the guiding structure 121. With this configuration, when the blood collection set according to the invention is not used, the syringe cover 2 shall cover around the injection needle 111 of the syringe 1, while the protruding spheres 221 on the rear end of the guiding tracks 22 that are provided on both sides of the syringe cover 2 are hold against the rear ends of the apertures 122 on the guiding structure 121. As the blood collection set is to be used, by pushing the syringe cover 2 backward, the syringe cover 2 can move by gliding its lateral guiding tracks 22 in apertures 122 on guiding structures 121 that are provided on the syringe 1, and this results in exposing the injection needle 111 for inserting into the targeting position of a subject. After completion of blood collection, it needs simply push the syringe cover 2 back to the original place, where, by means of the protruding spheres 221 provided at rear ends of lateral guiding track 22 on the syringe cover 2, and by virtue of fact that the outer diameter of the protruding spheres 221 is larger than those of apertures 122 on guiding structures 121, the guiding structure 121 can stop the protruding sphere 221 such that the syringe cover 2 can not be removed from the syringe 1.

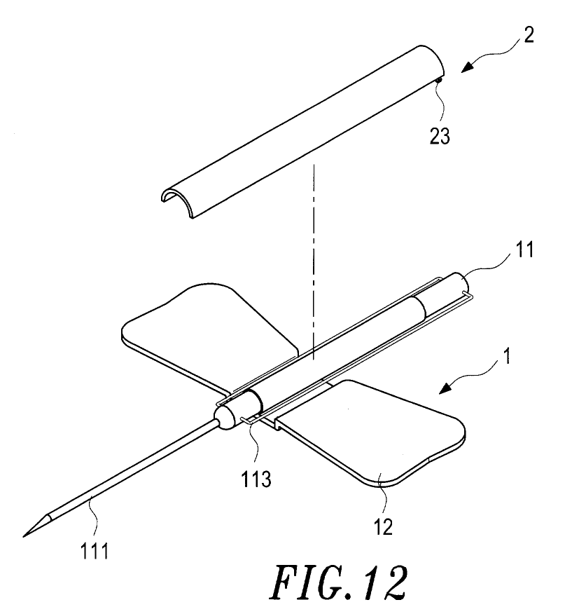

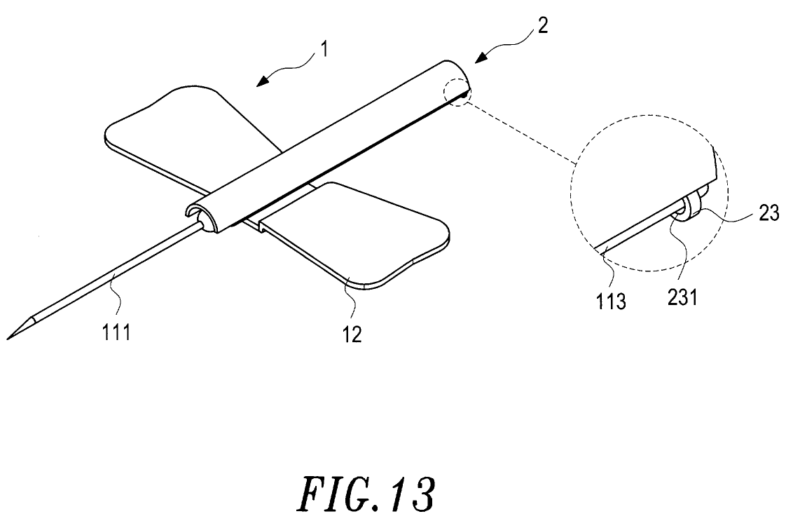

Referring to FIGS. 12 and 13, in another embodiment of the blood collection set according to the invention, on both sides of the syringe body 11 of the syringe 1, there is provided a guiding track 113, respectively. Further, on the both sides at the rear end of the base of the syringe cover 2, there is provided a protruding part 23 at a position corresponding to the position of the guiding track 113, wherein, on each protruding part 23, there is provided a through-hole 231 for the insertion of the gliding track 113. With this configuration, by gliding the aperture 122 on the protruding part 23 over the gliding track 113, the syringe cover 2 can move as gliding over the syringe 1.

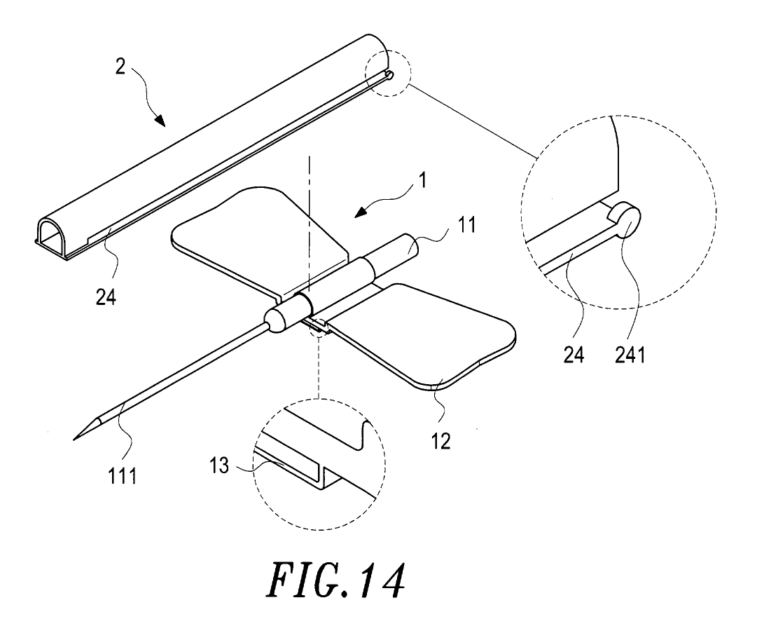

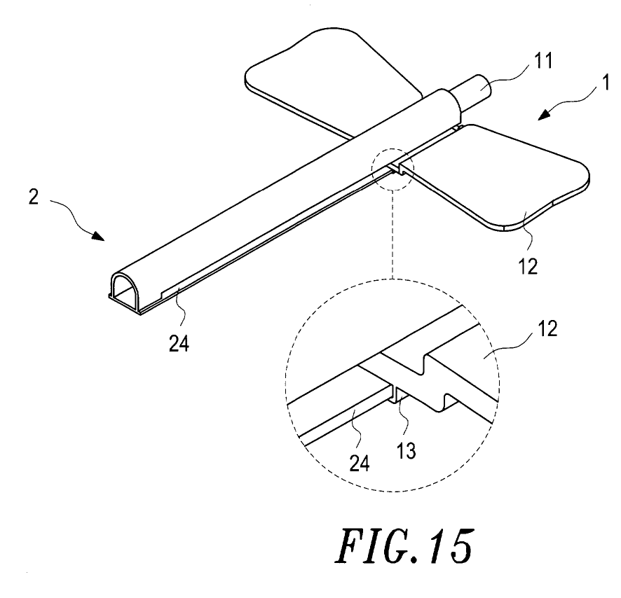

Referring to FIGS. 14 and 15, in still another embodiment of the blood collection set according to the invention, there is a guiding channel 13 provided on the base of the syringe 1, and also a guiding plate 24 provided on the base of the syringe cover 2. Further, there is provided one or more stop 241 on both sides of the rear end of the guiding plate 24. When the blood collection set according to the invention is to be used, by gliding the guiding plate 24 provided on the base of the syringe cover 2 through the guiding channel 13 on the syringe 1, the syringe cover 2 can displace back and forth over the syringe 1. Moreover, as the syringe cover 2 glides forth, the stop 241 provided on the rear end of the guiding plate 24 will stop against the rear end of the guiding channel 13 on the syringe 1 such that the syringe 1 can be prevent from detaching off the syringe cover 2.

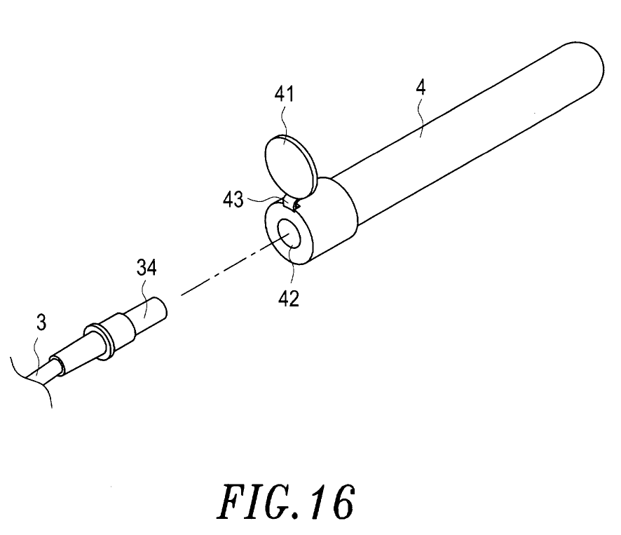

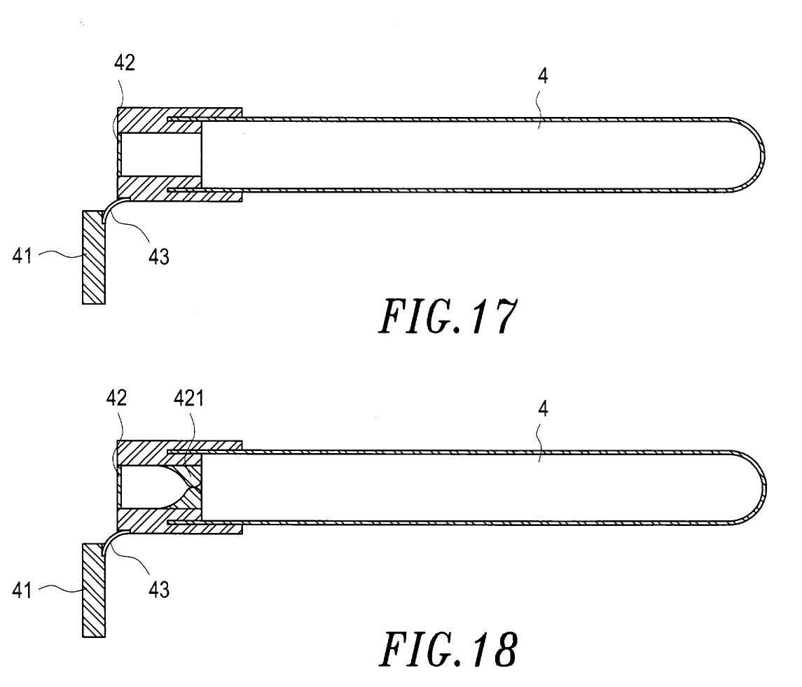

Referring to FIGS. 16, 17, and 18, in another embodiment of the blood collection set according to the invention, the plastic needle at the rear end of the flexible tube 3 can be substituted with a guiding tube 34. A membrane 42 is provided at the opening of the test tube 4. Further, the sidewall of the test tube 4 is linked to the test tube cap 41 via an elastic element 43. When the blood collection set according to the invention is to be used, the guiding tube 34 breaks through the membrane 42 to allow the blood flowing into the test tube 4. As the blood collection is complete and the guiding tube 34 is drawn out of the test tube 4, the membrane 42 that is broken by the guiding tube 34 is covered with the test tube cap 41 so as to prevent blood contamination from contacting with air. Moreover, on the inside wall at the opening of the test tube 4, a elastic material 421 is provided around the test tube 4 in a manner that, as the plastic needle 31 is drawn out of the test tube 4, the elastic material 421 can seal the test tube 4 and prevent the blood therein from contacting with the ambient air.

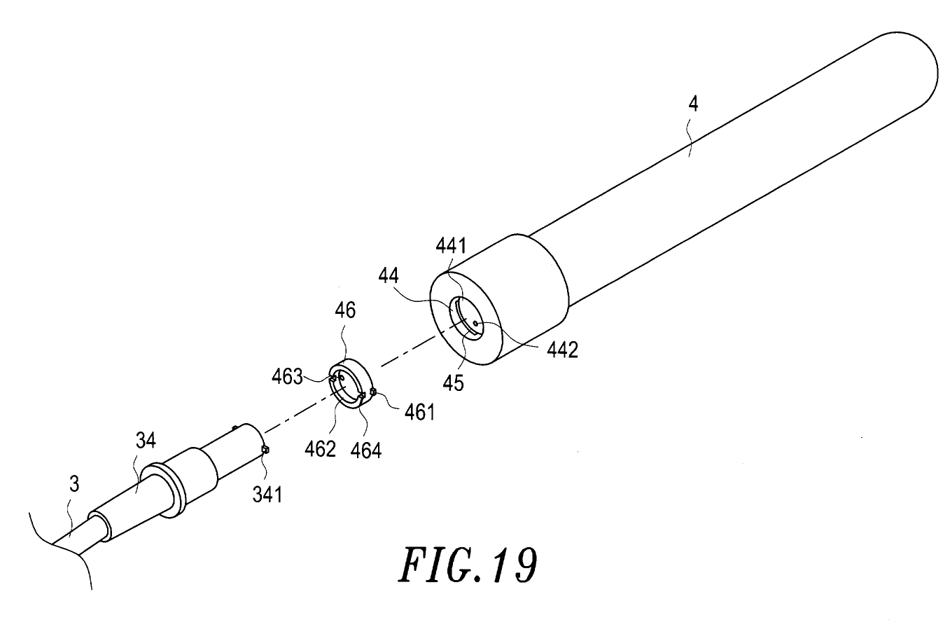

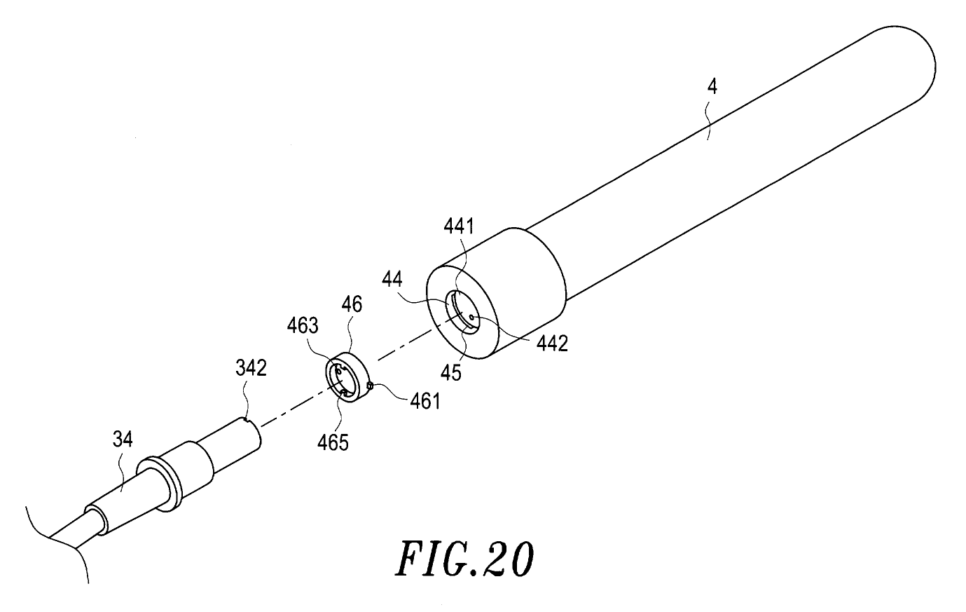

Now, referring to FIGS. 19 and 20, in another embodiment of the blood collection set according to the invention, at the opening of the test tube 4, an accommodating portion 44 can be provided having a separation plate 441 at its base. One or more guiding hole 442 is provided on the separation plate 441. Several grooves 45 are formed on the sidewall of the test tube 4 along the front edge of the separation plate 441. The accommodating portion 44 can accommodate a whirling element 46 whose outer edge is provided with protruding blocks 461 that can insert into the grooves 45. The whirling element 46 can whirl in the accommodating slot 322. An inserting slot 462 is provided concave at the front end of the whirling element 46. Several round holes 463 are provided at the base of the inserting slot 462 corresponding to the positions and number of the guiding holes 442 on the separating plate 441. Further, several clasping groove 464 are provided at the front edge of the whirling element 46. Also, several clasping bodies 341 are provided at the outer periphery of the guiding tube 34 corresponding to the position and the number of the clasping grooves 464. When the test tube 4 in the blood collection set according to the invention is not used, the guiding hole 442 of the test tube 4 and the round hole 463 of the whirling element 46 are in a state of not communicating with each other. In case of blood collection, by inserting the guiding tube 34 in the inserting slot 462 of the whirling element 46 and turning the guiding tube 34, the force produced by pushing the clasping body 341 against the clasping groove 464, the whirling element 46 can be pushed in a manner that the whirling element 46 can turn within a constant angle in associated with the pushing of the protruding block 461 on the outer edge of the whirling element 46 against the grooves 45. Thus, the round hole 463 of the whirling element 46 rotates to a state of being communicated with the guiding hole 442 of the test tube 4 such that the blood can flow through the round hole 463 and then the guiding hole 442 into the test tube 4. As the blood collection is accomplished, the whirling element 46 is rotated again by means of the guiding tube 34 such that the round hole 463 is not communicated with the guiding hole 442 again, where the guiding tube 34 can be drawn out of the test tube. Alternatively, on the sidewall of the test tube 4 at the front edge of the separation plate 441 can be provided convex with several protruding blocks, and, at the outer periphery of the whirling element 46, there are provided slots that can be placed in respectively for the protruding blocks. Further, no slot is provided on the sidewall of the test tube 4 at the front edge of the separation plate 441, as well as no protruding block is provided on the out periphery of the whirling element 46, and, in stead, the whirling element 46 can be allowed to whirl in the accommodating slot 322 by means of a thread. Furthermore, several clasping body 463 can be extended from the edge of the inserting slot 462 of the whirling element 46, and several clasping groove 342 can be provided at the outer periphery of the guiding tube 34 corresponding to the position and number of the individual clasping body 465.

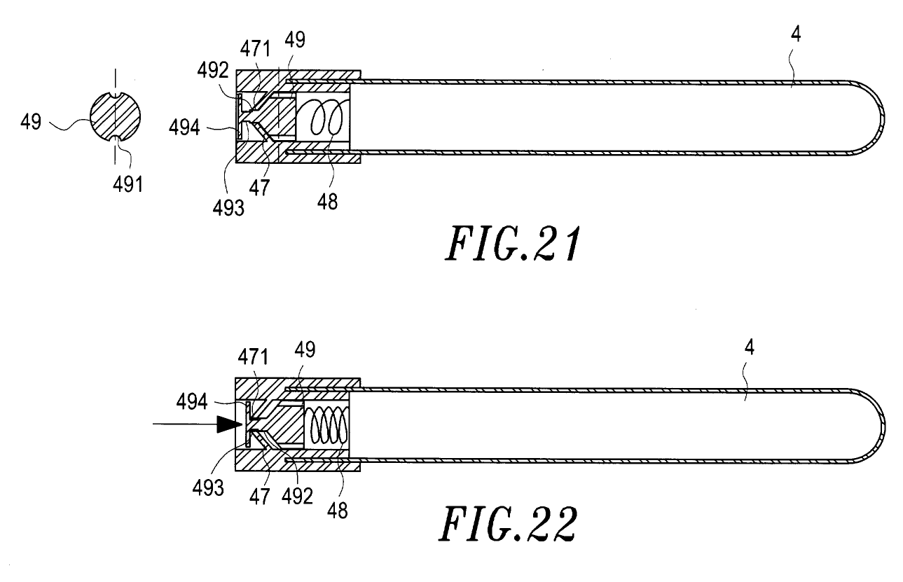

Referring to FIGS. 21 and 22, in another embodiment of the blood collection set according to the invention, a stopping block 47 is provided at the opening of the test tube 4, and at the center of the stopping block 47, there is provided a round hole 471. Further, in the test tube 4, a piston 49 made of an elastic material is provided by connecting through a spring 48. Several flow directing slot 491 are provided on the piston 49. The rear end of the piston 49 is accommodated in the round hole 471 of the stopping block 47 by means of a stopping piece 492. At the rear end of the stopping piece 492, there is provided a flow direction portion 493 having a outer diameter less than that of the stopping piece 492. By extending from outer edge of the flow direction portion 493, there is provided a stopping block 494. When the blood collection set according to the invention is not used, the spring 48 within the test tube 4 will push the piston 49 backward such that the stopping piece 492 will stop in the round hole 471 of the stopping block. As the guiding tube 34 pierces into the test tube 4, the guiding tube 34 will push the stopping block 494 of the piston 49 to enable the flow direction portion 493 of the piston 49 stay in the round hole 471. Further, since the outer diameter of the flow-directing portion 493 is less than that of the round hole 471, the blood can flow through the round hole 471 and the flow direction slot 491 of the piston into the test tube 4.

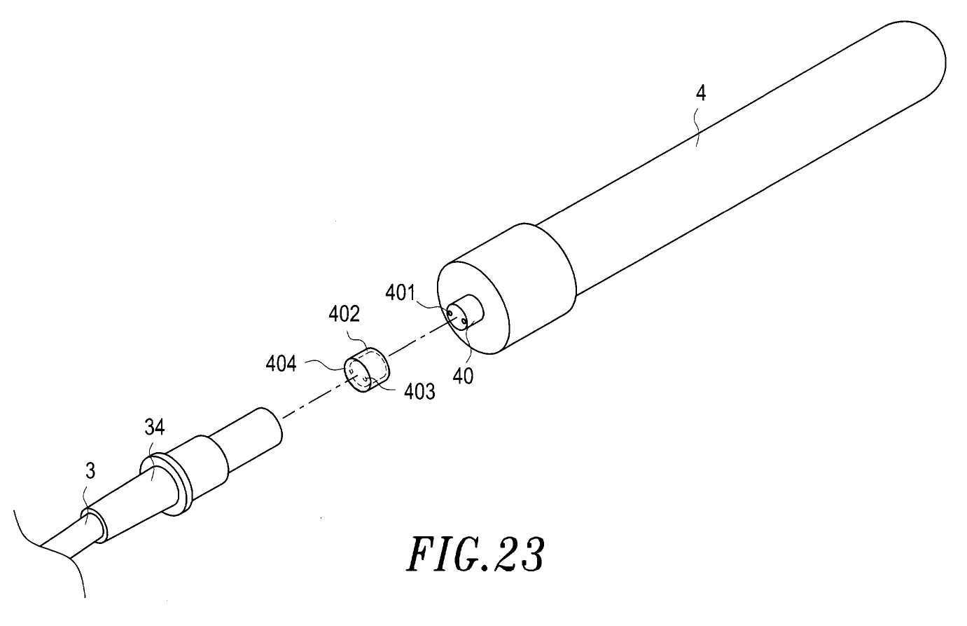

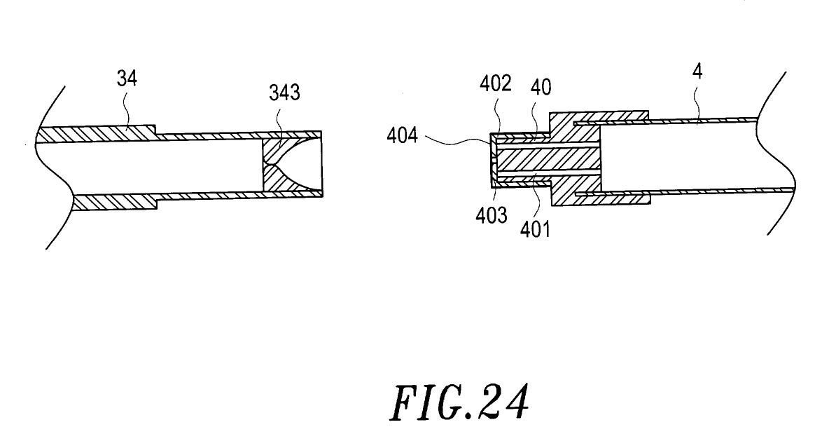

Referring to FIGS. 23 and 24, in another embodiment of the blood collection set according to the invention, a protrusion 40 is provided at the front edge of the test tube 4. One or more guiding hole 401 is provided on the protrusion 40. An outer mask 402 is provided to cover over the outer edge of the protrusion 40. Further, on the outer mask 402, several round holes 403 are provided corresponding to the position and number of guiding holes 401 on the protrusion 40. In addition, an annular protrusion 343 made of an elastic material is provided by lining around the inside wall of the guiding tube 34. When the blood collection set according to the invention is not used, the guiding hole 401 of the protrusion 40 and the round hole 403 of the outer mask 402 is not in a state of communication with each other. When the blood collection begins, upon engaging the test tube 4 with the guiding tube 34, the inside wall of the guiding tube 34 forces the thin membrane 404 of the test tube 4 to break such that the annular protrusion 343 of the guiding tube 34 will enclose over the outer edge of the outer mask 402 of the test tube 4. Next, by rotating the guiding tube 34, a friction force is produced due to the contact between the outer mask 402 and the annular protrusion 343 of the guiding tube 34. By means of this friction force, the annular outer mask 402 will rotate on the protrusion 40 into a condition that the round hole 403 communicates with the guiding hole 401 and hence the blood flows into the test tube 4 via the guiding tube 34.

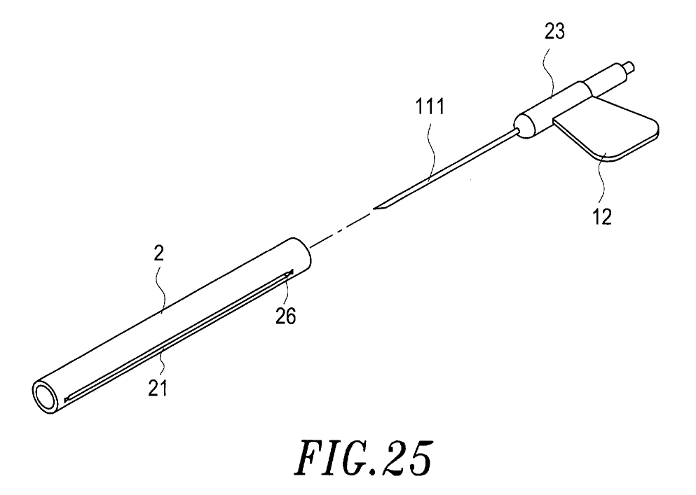

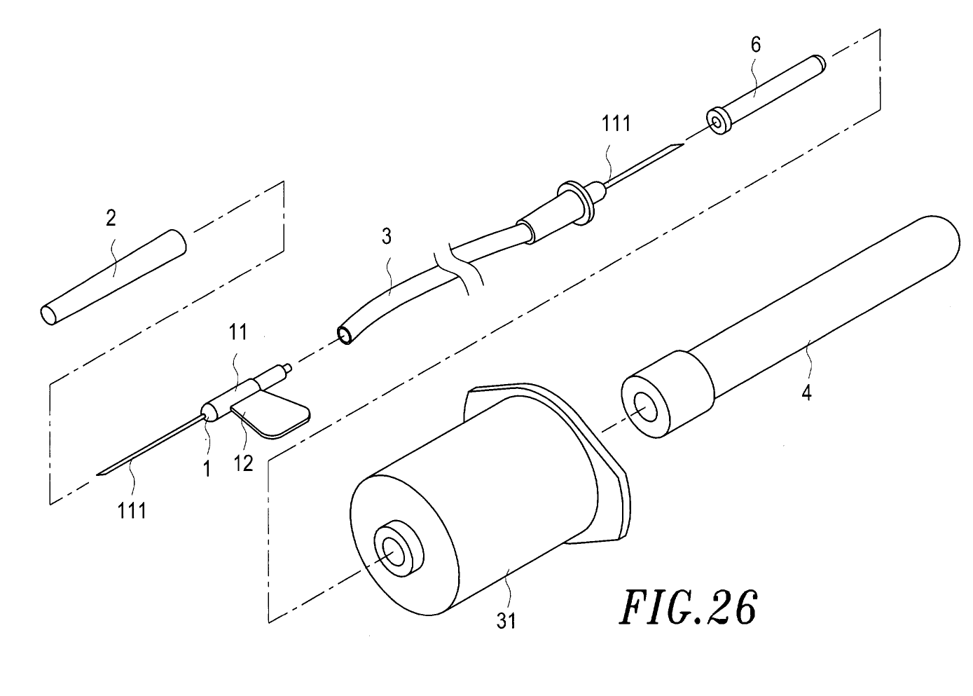

Referring to FIG. 25, in another embodiment of the blood collection set according to the invention, only one lateral piece 12 is extended from a single side of the syringe 1. A gliding slot 21 is provided on one side of the syringe cover 2 corresponding to the position of the lateral piece 12. A clasping portion 26 is provided convex on each of the front and rear end of the gliding slot 21 such that, as the syringe cover 2 moves to the front end or the rear end of the syringe 1, the syringe cover 2 could be fixed on the lateral piece 12 of the syringe 1 by means of said clasping portion 26 and hence the syringe cover 2 will stay fixed at the front end or the rear end of the syringe 1.

The blood collection set provided by the invention has several following advantages over the conventional prior art:

1. In the blood collection set according to the invention, by means of the engagement of fasteners with the syringe cover, the syringe can be used in combination with the syringe cover.

2. By combining the syringe with the syringe cover and by replacing a plastic needle for the conventional injection needle connected at the rear end of the flexible tube, the medical practitioner in operation or the cleanser in cleansing can avoid the risk of being hurt and/or infection.

Many changes and modifications in the above-described embodiment of the invention can, of course, be carried out without departing from the scope thereof. Accordingly, to promote the progress in science and the useful arts, the invention is disclosed and is intended to be limited only by the scope of the appended claims.

What is claimed is:

1. A blood collection set comprises:
a syringe, provided with several lateral pieces each extending from the base of the syringe body, respectively, wherein said syringe body is fitted with an injection needle at the front end thereof, and is provided protrudingly with one or more fasteners at the top thereof;
a syringe cover, provided with several gliding slots thereon corresponding to the number and positions of said fasteners on said syringe, wherein on both of the front and rear ends of said gliding slots, a protruding button is provided in a manner that a fastening portion is formed on the front end and the rear end of said gliding slots, respectively, and wherein the base of the syringe cover is flat and is engaged with said lateral pieces of the syringe;
a flexible tube, the front end of said flexible tube being connected with the rear end of said syringe, while the rear end of said flexible tube being connected with a plastic needle, wherein on said flexible tube, a interposing structure is sleeved on; and
a test tube, provided with a test tube cap that is made of an elastic material and is sleeved on the opening of said test tube;
wherein said syringe is positioned within said gliding slot on top of said syringe cover via said fastener, and by means of said gliding slot, said syringe cover can gliding over said syringe, and wherein, as said syringe cover gliding to the front end or the rear end of said syringe, said protruding button on the front end and the rear end of said gliding slot can stop said fasteners in said fastening portion, while said plastic needle provided on the rear end of said flexible tube can puncture into said test tube cap.

2. A blood collection set as recited in claim 1, wherein the front end of the syringe cover can be connected with a cap by means of a connecting element.

3. A blood collection set as recited in claim 1, wherein said fasteners is a spherical body provided on the top of a cylindrical post in a form that the outer diameter of said spherical body is larger than that of said cylindrical post, and wherein said fastener move in said gliding slot by means of said cylindrical post, while said spherical bodies is clasped movably within said gliding slot such that said syringe cover will not detach from said syringe.

4. A blood collection set as recited in claim 1, wherein said fastener is provided on any position on the periphery of said syringe body.

5. A blood collection set as recited in claim 1, wherein, in stead of providing fasteners on the top of said syringe, a guiding structure is provided on the front end of said lateral pieces near both sides of said syringe body, respectively, and, alternatively, on both sides of said syringe cover, there are provided guiding tracks that extend backwardly therefrom and through the aperture of said guiding structure, and wherein, a protruding sphere is provided on the rear end of each of said guiding track.

6. A blood collection set as recited in claim 1, wherein, in stead of providing fasteners on the top of said syringe, guiding tracks is provided on both sides of said syringe body, and alternatively, in stead of providing gliding slots, on both sides of the base of said syringe cover, there is provided a protruding part at a position corresponding to the position of said guiding track, respectively, and wherein, on each protruding part, there is provided a through-hole for the insertion of said gliding track.

7. A blood collection set as recited in claim 1, wherein, in stead of providing fasteners on the top of said syringe, guiding channel is provided on the base of said syringe body, and alternatively, in stead of providing gliding slot, on the base of said syringe cover, there is provided guiding plate for inserting in said guiding channel, and wherein, on the rear end of said guiding plate, there is provided one or more stops.

8. A blood collection set as recited in claim 1, wherein only one lateral piece is extended from a single side of the syringe 1, a gliding slot is provided on one side of the syringe cover corresponding to the position of the lateral piece, and a clasping portion is provided convex on each of the front and rear end of the gliding slot.

9. A blood collection set as recited in claim 1, wherein said interposing structure is consisted of a main body and a holding element, wherein, on both sides of said holding element, a shaft lever is extended therefrom; wherein, within said main body, there is provided concave an accommodating slot that has a slope for said flexible tube to extend through, and on each of both wall surfaces of said accommodating slot, there is provided a moving slot for positioning of said shaft lever of said holding element.

10. A blood collection set as recited in claim 1, wherein said interposing structure provided on said flexible tube is a clipping element.

11. A blood collection set as recited in claim 1, wherein said plastic needle at the rear end of said flexible tube can be substituted with a guiding tube, and wherein a membrane is provided at the opening of said test tube, and the sidewall at the opening of said test tube is linked to said test tube cap via an elastic element.

12. A blood collection set as recited in claim 11, wherein, on the inside wall of said test tube, an elastic material is provided around said test tube.

13. A blood collection set as recited in claim 11, wherein a stopping block is provided in the opening of said test tube, and at the center of said stopping block, there is provided a round hole, and, in said test tube, a piston made of an elastic material is provided by connecting through a spring, wherein several flow directing slot are provided on said piston, and wherein the rear end of said piston is accommodated in said round hole of said stopping block by means of a stopping piece, and wherein, at the rear end of said stopping piece, there is provided a flow direction portion having a outer diameter less than that of said stopping piece, and, a stopping block is provided by extending from outer edge of said flow direction portion.

14. A blood collection set as recited in claim 11, wherein a protrusion is provided at the front edge of said test tube, and one or more guiding hole is provided on said protrusion, and wherein an outer mask is provided to cover over the outer edge of said protrusion, and, on said outer mask, several round holes are provided corresponding to the position and number of guiding holes 401 on said protrusion, and in addition, an annular protrusion made of an elastic material is provided by lining around the inside wall of said guiding tube.

15. A blood collection set as recited in claim 11, wherein at the opening of said test tube, an accommodating portion is provided having a separation plate at its base, and one or more guiding hole is provided on said separation plate, wherein several grooves are formed on the sidewall of said test tube along the front edge of said separation plate, wherein said accommodating portion can accommodate a whirling element whose outer edge is provided with protruding blocks that can insert into said grooves, wherein said whirling element can whirl in said accommodating slot, wherein an inserting slot is provided concave at the front end of said whirling element, and several round holes are provided at the base of said inserting slot corresponding to the positions and number of said guiding holes on said separating plate, wherein several clasping groove are provided at the front edge of said whirling element, and wherein several clasping bodies are provided at the outer periphery of said guiding tube corresponding to the position and the number of the clasping grooves.

16. A blood collection set as recited in claim 15, wherein on the sidewall of said test tube at the front edge of said separation plate is provided convex with several protruding blocks, and wherein, at the outer periphery of said whirling element, there are provided slots that can be placed in respectively for said protruding blocks.

17. A blood collection set as recited in claim 15, wherein no slot is provided on the sidewall of said test tube at the front edge of said separation plate, as well as no protruding block is provided on the out periphery of said whirling element, and, in stead, said whirling element is allowed to whirl in said accommodating slot by means of a thread.

18. A blood collection set as recited in claim 15, wherein several clasping bodies are extended from the edge of said inserting slot of said whirling element, and wherein several clasping groove are provided at the outer periphery of said guiding tube corresponding to the position and number of said individual clasping body.
